(19) United States
(12) Patent Application Publication
(10) Pub. No.: US 2004/0248139 A1
(43) Pub. Date: Dec. 9, 2004

(54) Eit-6, a polypeptide encoded by an estrogen regulated gene

(76) Inventors: Kornelia Polyak, Brookline, MA (US); Pankaj Seth, Boston, MA (US)

Correspondence Address: FISH & RICHARDSON P.C., CITIGROUP CENTER 52ND FLOOR, 153 EAST 53RD STREET, NEW YORK, NY, 10022-4611, US

(21) Appl. No.: 10/494,930

(22) PCT Filed: Nov. 8, 2002
(86) PCT No.: PCT/US02/35899

Related U.S. Application Data
(60) Provisional application No. 60/337,754, filed on Nov. 9, 2001.

(51) Int. Cl. C12Q 1/68; G01N 33/53; G01N 33/567; G01N 33/574

(52) U.S. Cl. 435/6; 435/7.2

(57) ABSTRACT

The invention features isolated DNA molecules encoding EIT-6, vectors containing the DNA, cells containing the vectors, and the isolated EIT-6 molecules. Also featured by the invention are methods of inhibiting the activity and expression of EIG-6

### TECHNICAL FIELD

This invention relates to genes regulated by estrogen and tamoxifen, and more particularly to the gene encoding EIT-6.

### BACKGROUND

The action of estrogen is mediated by estrogen receptors (ER) α and β which are members of the nuclear hormone receptor family [Katzenellenbogen (1996) Biol. Reprod. 54:5582-5591; Mangelsdorf et al. (1995) Cell 83:835-839]. While estrogen is known to have mitogenic activity, the molecular mechanism of this mitogenic activity is not known.

### SUMMARY

The invention is based on the (a) identification of a gene (the EIT-6 gene) the transcription of which is activated by estrogen and (b) the finding that the EIT-6 protein encoded by the EIT-6 gene enhances proliferation or survival of cells, e.g., breast cancer cells. Thus the invention features a DNA encoding EIT-6 protein, the EIT-6 protein (sometimes referred to herein as “EIT-6”), a vector containing the DNA, cells containing the vector, a method of making the EIT-6 protein, and an antibody that binds to the EIT-protein. The invention also includes methods of inhibiting the activity of EIT-6, methods of inhibiting expression of EIT-6, and methods of identifying compounds that inhibit the activity of EIT-6.

More specifically, the invention features a method of identifying a compound that inhibits an activity of EIT-6. The method involves: (a) identifying a cell as expressing a functional EIT-6 molecule; and (b) testing in vitro for the ability of a test compound to inhibit proliferation or survival of the cell. A compound that inhibits the proliferation or survival of the cell is a compound that can potentially inhibit the activity of EIT-6. The cell can be transfected with or transformed with a DNA encoding a functional EIT-6 molecule. The method can include the additional steps of: (c) providing a second cell that does not substantially express a functional EIT-6 molecule; and (d) determining in vitro if the test compound inhibits proliferation or survival of the second cell.

Another embodiment of the invention is a method of identifying a compound that inhibits an activity of EIT-6. The method involves contacting a test compound with a functional EIT-6 molecule and determining whether the test compound inhibits an activity of the functional EIT-6 molecule. The activity can include hydroxylation of a proline residue in a polypeptide or conversion of 2-ketoglutarate to succinate.

Also encompassed by the invention is a method of inhibiting an activity of EIT-6. The method involves: (a) identifying a cell as expressing a functional EIT-6 molecule; and (b) contacting the cell with a compound that inhibits an activity of EIT-6. Also provided is a method of inhibiting the activity of EIT-6 in a subject. The method involves: (a) identifying a mammal as having a cancer that expresses EIT-6; and (b) administering to the mammal a compound that inhibits an activity of EIT-6. In both these methods the activity can include hydroxylation of a proline residue in a polypeptide or conversion of 2-ketoglutarate to succinate and the compound can be a 2-oxoglutarate analog (e.g., N-oxalylglycine, dimethyl-oxalylglycine, or N-oxalyl-2S-alanine), pyridine-2,5-dicarboxylic acid, or an analog of pyridine-2,5-dicarboxylic acid. In the method of inhibiting the activity of EIT-6 in a subject, the cell can be in a mammalian subject. The cell can be a cancer cell, e.g., a breast cancer cell. Furthermore, the cell can be an estrogen responsive cell.

Another aspect of the invention is an isolated DNA that contains: (1) a nucleic acid that encodes a polypeptide consisting of (a) SEQ ID NO:1 or (b) SEQ ID NO:1 but with one or more conservative substitutions;. or (2) the complement of the nucleic acid. The polypeptide has the ability to enhance the proliferation of a cell. In a particular embodiment, the nucleic acid consists of SEQ ID NO:2. Also included in the invention is a vector containing any DNA of the invention. In the vector, the nucleic acid can optionally be operably linked to a transcriptional regulatory element (TRE). Yet another aspect of the invention is a cell (e.g., a eukaryotic cell or a prokaryotic cell) containing any vector of the invention. The invention features a method of making a polypeptide. The method involves (a) culturing a cell containing a vector of the invention in which the nucleic acid encoding the polypeptide is operably linked to a TRE and (b) isolating the polypeptide from the culture. Also embraced by the invention is (a) a polypeptide encoded by any DNA of the invention and (b) a polypeptide that contains (i) a fragment of the polypeptide consisting of SEQ ID NO:1 or (ii) the fragment but with one or more conservative substitutions. The fragment and the fragment with one or more conservative substitutions each have the ability to enhance proliferation of a cell. Another embodiment of the invention is a DNA that contains a nucleic acid sequence that encodes (a) a fragment of a polypeptide consisting of SEQ ID NO:1 or (b) the fragment but with one or more conservative substitutions. The fragment and the fragment with one or more conservative substitutions each are at least 85 amino acids long and each have the ability to enhance proliferation of a cell.

The invention features a method of inhibiting expression of EIT-6 in a cell. The method involves delivery to the inside of a cell of an antisense oligonucleotide that hybridizes to an EIT-6 transcript, wherein the antisense oligonucleotide inhibits expression of EIT-6 in the cell. The antisense oligonucleotide can be an RNA antisense oligonucleotide. The cell can be in a mammal. In this method delivery can be by introduction into the cell of the antisense oligonucleotide or by introduction into the cell of a nucleic acid comprising a TRE operably linked to a nucleic acid sequence, the nucleic acid sequence being transcribed in the cell into the antisense oligonucleotide. The cell can be a cancer cell, e.g., a breast cancer cell.

The invention also encompasses an antibody that binds to EIT-6. The antibody can be a monoclonal antibody or a polyclonal antibody.

Another feature of the invention is a method of identifying a compound that inhibits an activity of EIT-6. The method involves: (a) providing a cell that is transfected with or transformed with a DNA encoding a functional EIT-6 molecule; and (b) testing for the ability of a test compound to inhibit proliferation or survival of the cell; a compound that inhibits the proliferation or survival of the cell is a compound that can potentially inhibit the activity of EIT-6.

“Polypeptide” and “protein” are used interchangeably and mean any peptide-linked chain of amino acids, regardless of length or post-translational modification. The invention also features EIT-6 polypeptides with conservative substitutions. Conservative substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine.

The term “isolated” polypeptide or peptide fragment as used herein refers to a polypeptide or a peptide fragment which either has no naturally-occurring counterpart or has been separated or purified from components which naturally accompany it, e.g., in tissues such as pancreas, liver, spleen, ovary, testis, muscle, joint tissue, neural tissue, gastrointestinal tissue or tumor tissue, or body fluids such as blood, serum, or urine. Typically, the polypeptide or peptide fragment is considered “isolated” when it is at least 70%, by dry weight, free from the proteins and other naturally-occurring organic molecules with which it is naturally associated. Preferably, a preparation of a polypeptide (or peptide fragment thereof) of the invention is at least 80%, more preferably at least 90%, and most preferably at least 99%, by dry weight, the polypeptide (or the peptide fragment thereof), respectively, of the invention. Thus, for example, a preparation of polypeptide x is at least 80%, more preferably at least 90%, and most preferably at least 99%, by dry weight, polypeptide x. Since a polypeptide that is chemically synthesized is, by its nature, separated from the components that naturally accompany it, the synthetic polypeptide is “isolated.”

An isolated polypeptide (or peptide fragment) of the invention can be obtained, for example, by extraction from a natural source (e.g., from tissues); by expression of a recombinant nucleic acid encoding the polypeptide; or by chemical synthesis. A polypeptide that is produced in a cellular system different from the source from which it naturally originates is “isolated,” because it will necessarily be free of components which naturally accompany it. The degree of isolation or purity can be measured by any appropriate method, e.g., column chromatography, polyacrylamide gel electrophoresis, or HPLC analysis.

An “isolated DNA” is either (1) a DNA that contains sequence not identical to that of any naturally occurring sequence, or (2), in the context of a DNA with a naturally-occurring sequence (e.g., a cDNA or genomic DNA), a DNA free of at least one of the genes that flank the gene containing the DNA of interest in the genome of the organism in which the gene containing the DNA of interest naturally occurs. The term therefore includes a recombinant DNA incorporated into a vector, into an autonomously replicating plasmid or virus, or into the genomic DNA of a prokaryote or eukaryote. The term also includes a separate molecule such as: a cDNA where the corresponding genomic DNA has introns and therefore a different sequence; a genomic fragment that lacks at least one of the flanking genes; a fragment of cDNA or genomic DNA produced by polymerase chain reaction (PCR) and that lacks at least one of the flanking genes; a restriction fragment that lacks at least one of the flanking genes; a DNA encoding a non-naturally occurring protein such as a fusion protein, mutein, or fragment of a given protein; and a nucleic acid which is a degenerate variant of a cDNA or a naturally occurring nucleic acid. In addition, it includes a recombinant nucleotide sequence that is part of a hybrid gene, i.e., a gene encoding a non-naturally occurring fusion protein. Also included is a recombinant DNA that includes a portion of SEQ ID NO:2. It will be apparent from the foregoing that isolated DNA does not mean a DNA present among hundreds to millions of other DNA molecules within, for example, cDNA or genomic DNA libraries or genomic DNA restriction digests in, for example, a restriction digest reaction mixture or an electrophoretic gel slice.

As used herein, a “functional EIT-6 molecule” is (a) a full-length, wild-type EIT-6; (b) a functional fragment of EIT-6; or (c) (a) of (b) but with not more than (i.e., not more than, for example, 50, 45, 40, 35, 30, 25, 20, 17, 14, 12, 10, nine, eight, seven, six, five, four, three, two, or one) conservative substitution(s). Functional EIT-6 molecules with one or more conservative substitutions have at least 10% (e.g., 10%, 20%, 30%, 40%, 50%, 60%, 70%, 80%, 90%, 95%, 98%, 99%, 100%, or more) of the ability of wild-type, full-length EIT-6 to enhance the proliferation or survival of T47D breast cancer cells when tested as described in Example 3.

As used herein, a “functional fragment” of an EIT-6 polypeptide is a fragment of the polypeptide that is shorter than the full-length polypeptide and has at least 10% (e.g., 10%, 20%, 30%, 40%, 50%, 60%, 70%, 80%, 90%, 95%, 98%, 99%, 100%, or more) of the ability of the full-length polypeptide to enhance the proliferation or survival of T47D breast cancer cells when tested as described in Example 3. Fragments of interest can be made either by recombinant, synthetic, or proteolytic digestive methods. Such fragments can then be isolated and tested for their ability to enhance the proliferation or survival of T47D breast cancer cells as measured by [³H]-thymidine incorporation or cell or colony counting.

As used herein, “operably linked” means incorporated into a genetic construct so that expression control sequences effectively control expression of a coding sequence of interest.

As used herein, the term “antibody” refers not only to whole antibody molecules, but also to antigen-binding fragments, e.g., Fab, F(ab′)₂, Fv, and single chain Fv (ScFv) fragments. Also included are chimeric antibodies.

As used herein, “prophylaxis” can mean complete prevention of the symptoms of a disease, a delay in onset of the symptoms of a disease, or a lessening in the severity of subsequently developed disease symptoms. “Prevention” should mean that symptoms of the disease (e.g., cancer) are essentially absent. As used herein, “therapy” can mean a complete abolishment of the symptoms of a disease or a decrease in the severity of the symptoms of the disease. As used herein, a “protective” immune response is an immune response that is prophylactic and/or therapeutic.

Unless otherwise defined, all technical and scientific terms used herein have the same meaning as commonly understood by one of ordinary skill in the art to which this invention pertains. In case of conflict, the present document, including definitions, will control. Preferred methods and materials are described below, although methods and materials similar or equivalent to those described herein can be used in the practice or testing of the present invention. All publications, patent applications, patents and other references mentioned herein are incorporated by reference in their entirety. The materials, methods, and examples disclosed herein are illustrative only and not intended to be limiting.

Other features and advantages of the invention, e.g., inhibiting growth of cancer cells, will be apparent from the following description, from the drawings and from the claims.

### DESCRIPTION OF DRAWINGS

FIG. 1A is a diagram comparing data in a photograph of northern blots (left panel) to data obtained by SAGE (right panel). RNA from untreated (“C”), estrogen-treated (“E”), and tamoxifen-treated (“T”) ZR75-1 breast cancer cells was subjected to northern blot analysis using radiolabeled cDNA probes corresponding to the genes indicated in the column to the left of the photograph. The same RNA samples were also subjected to SAGE and the numbers in the columns indicate the relative number of SAGE tags (corresponding to the genes indicated in the column to the left of the photograph) in SAGE libraries generated from the three RNA samples. CATH D, cathepsin gene; PR, progesterone receptor gene; ND, not detected.

FIG. 1B is a photograph of Northern blots of RNA from the indicated breast cancer lines that were either not treated (“C”), estrogen-treated (“E”), or tamoxifen-treated (“T”). Expression (“+”) and lack of expression (“−”) of estrogen receptor a by the cell lines used is indicated in the right column.

FIG. 1C is a photograph of Northern blots of RNA from BT474 breast cancer cells that had not been treated (“C”) or had been treated with estrogen (“E”), tamoxifen (“T”), ICI 128,780 (“IC”), a combination of estrogen and tamoxifen (“T+E”), or a combination of estrogen and ICI 128,780 (“ICI+E”). The Northern blots were exposed to radiolabeled EIT-6, cathepsin D (“CATH D”) or β-actin (“β-ACTIN”) cDNA probes.

FIG. 2A is a photograph of Northern blots of RNA from ZR75-1 breast cancer cells ZR-75-1 that had been treated with estrogen for the indicated number of hours prior to RNA isolation. The northern blots were exposed to radiolabeled EIT-6, cathepsin D (“CATH D”) or β-actin (“β-ACTIN”) cDNA probes.

FIG. 2B is a photograph of Northern blots of RNA from BT-474 breast cancer cells that had been treated with ethanol (solvent) only (“C”), ethanol and cycloheximide (“C+C”), ethanol and estrogen (“E”), or ethanol, estrogen, and cycloheximide (“E+C”). The Northern blots were exposed to radiolabeled EIT-6, cathepsin D (“CATH D”) or β-actin (“β-ACTIN”) cDNA probes.

FIG. 2C is a bar graph showing the results of a luciferase assay of cells transiently transfected with an expression construct encoding estrogen receptor a and with a construct in which the luciferase gene was operably linked to a promoter region sequence (˜5.5 kb) of the EIT-6 gene. The cells were either untreated (“UT”) or treated with estrogen (“E”) after transfection. The graph shows data from two separate experiments (“Exp. 1” and “Exp. 2”).

FIG. 2D is a series of diagrams (i) depicting the nucleotide sequences of four (E1-E4) putative estrogen response elements (ERE) (within the promoter region sequence (˜5.5 kb) referred to in the description of FIG. 2C) aligned for optimum homology with the ERE consensus sequence (top panel), (ii) showing maps of the EIT-6 gene and of reporter constructs used to test concatamers of the four putative ERE for estrogen-responsiveness (bottom panel). In the map of the EIT-6 gene (top map), exons are indicated by the open boxes numbered E1-E4 and the transcription start site is indicated by an arrow. Nucleotide numbering is with reference to the transcription start site (“+1”). All the reporter constructs contained the luciferase coding sequence, a TATA box, and concatemers containing different combinations and numbers of copies of the four putative EREs. The triangles indicate the orientation of the control and putative ERE; triangles with apices to the right indicate that the orientation of the relevant ERE is as it occurs in the EIT-6 gene and the triangle with the apex to the left indicates that orientation of the relevant ERE is opposite to the orientation in which it occurs in the EIT-6 gene. Control constructs contained either no ERE (“TATA”) or a concatamer of two copies of an ERE (“E”) from the vitollogenin promoter (“VIT”). The designations of the various constructs are shown in the right column of the bottom panel. (putative ERE E1, SEQ ID NO:3; putative ERE E2, SEQ ID NO:4; putative ERE E3, SEQ ID NO:5; putative ERE E4, SEQ ID NO:6; ERE consensus sequence, SEQ ID NO:7).

FIG. 2E is a bar graph showing the results of a luciferase assay of HepG2 cells transiently transfected with an expression construct encoding estrogen receptor a, one of each of the constructs described in the above description of FIG. 2D, and an expression vector containing the β-galactosidase coding sequence under control of a constitutive promoter. Data are expressed as “fold induction by estrogen” and are means of values obtained from three independent experiments. Luciferase activity was normalized for transfection efficiency by calculating the ratio of luciferase activity to β-galactosidase activity.

FIG. 3A is a diagram showing the amino acid sequences of segments in C-terminal regions of human EIT-6 (amino acids 260-383) (SEQ ID NO:8), rat SM20 (amino acids 214-336) (SEQ ID NO:9), and C. elegans egl-9 (amino acids 451-574) (SEQ ID NO:10) aligned for maximum homology. The alignment was derived using the MacVector program and ClustalW alignment. Homologies are indicated by boxes and consensus amino acids are shown below the human EIT-6 sequence.

FIG. 3B is a diagram showing a phylogenetic comparison of human EIT-6 and a variety of homologues.

FIG. 3C is a series of fluorescence micrographs of MCF10A transiently transfected with expression vectors encoding green fluorescence protein (GFP) (“GFP”), GFP fused to a protein that localizes to mitochondria (“mito-GFP”), or GFP fused to EIT-6 (“EIT-6-GFP”).

FIG. 3D is a bar graph showing the numbers of colonies that were obtained after drug (hygromycin) selection (for 2 weeks) of cells that had been transfected with either a control expression vector (“pCEP4”) or an expression vector containing cDNA encoding human EIT-6 fused at its C-terminus to a double hemagglutinin tag (“pCEP4-EIT6”). In one experiment (left two bars), culture medium containing normal fetal bovine serum (FBS) (“Regular serum”) was used. In the other experiment (right four bars) culture medium containing charcoal/dextran-treated (i.e., estrogen depleted) FBS (“Charcoal/dextran treated serum”) was used. Data are expressed as “colony numbers/cm²”.

FIG. 4A is a depiction of the amino acid sequence of human EIT-6 (SEQ ID NO:1).

FIG. 4B is a depiction of the nucleotide sequence of cDNA (SEQ ID NO:2) encoding human EIT-6.

FIG. 5A is a depiction of the chemical structures of (a) pyridine-2,5-dicarboxylic acid (a) and pyridine-2,5-dicarboxylic acid analogues that inhibit hydroxylation of proline residues by prolyl hydroylase (b-d).

FIG. 5B is a depiction of the chemical structures of pyridine-2,5-dicarboxylic acid analogues (a-j) in each of which the 5-carboxylic group has been replaced by one of a variety of acyl sulfonamide groups.

### DETAILED DESCRIPTION

The inventors have by SAGE (serial analysis of gene expression) identified 61 genes that are regulated by estrogen and 15 genes that are regulated by tamoxifen in an estrogen-responsive (ER+) human breast cancer cell line; this finding was, for some of these genes, confirmed by northern blot analysis. One of the genes identified was the EIT-6 (estrogen-induced tag 6) gene. Because of the relative abundance in SAGE libraries prepared from various hormone-responsive tissues of a SAGE tag corresponding to EIT-6 mRNA, the inventors decided to study the EIT-6 gene in greater detail.

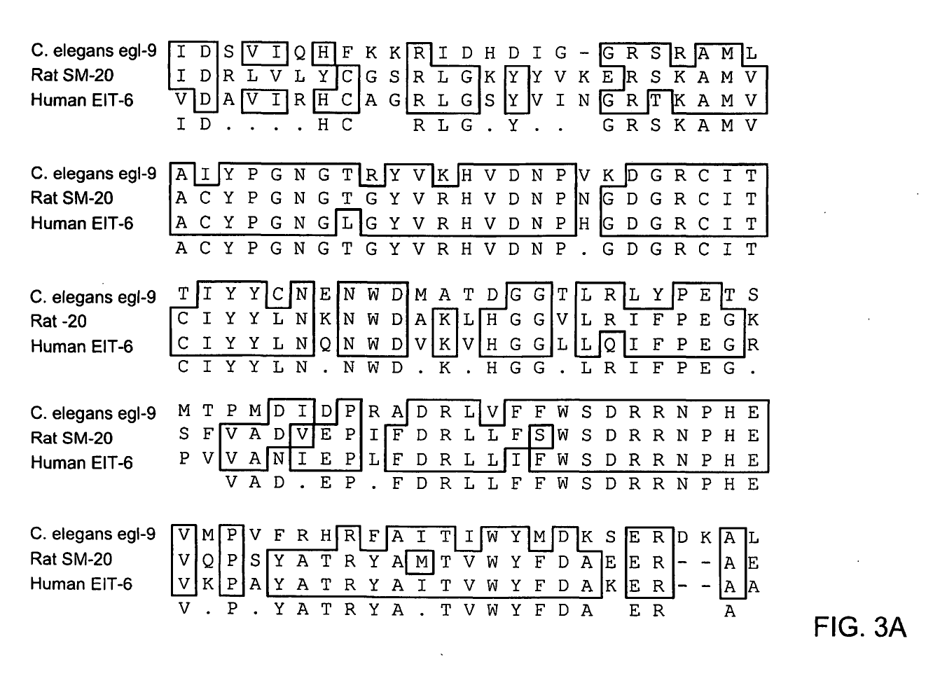

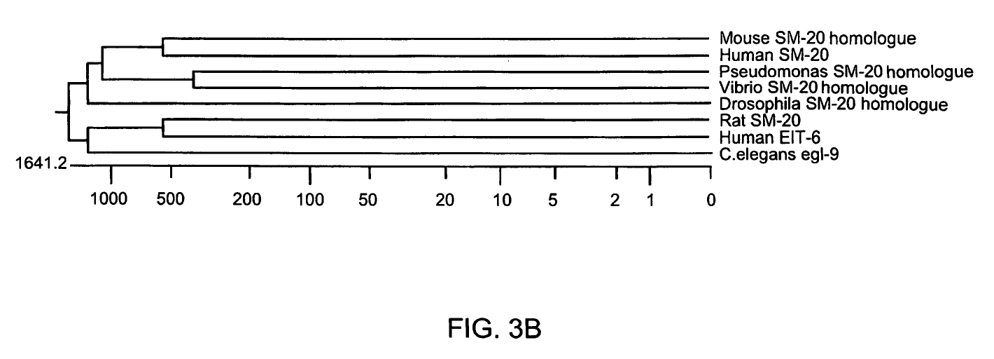

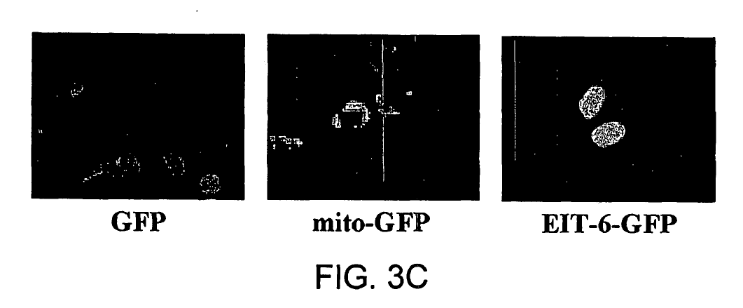

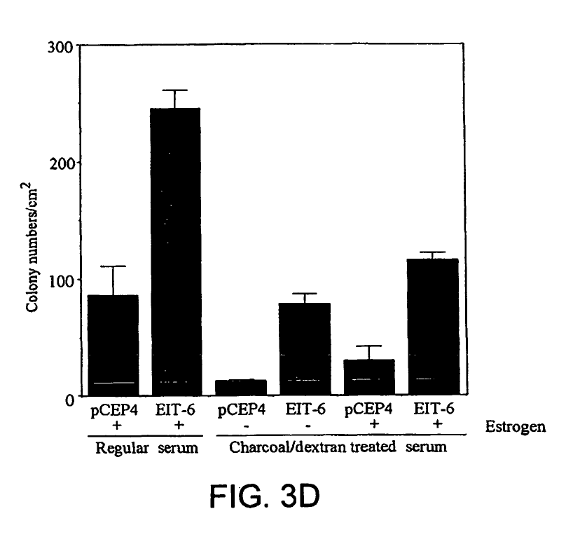

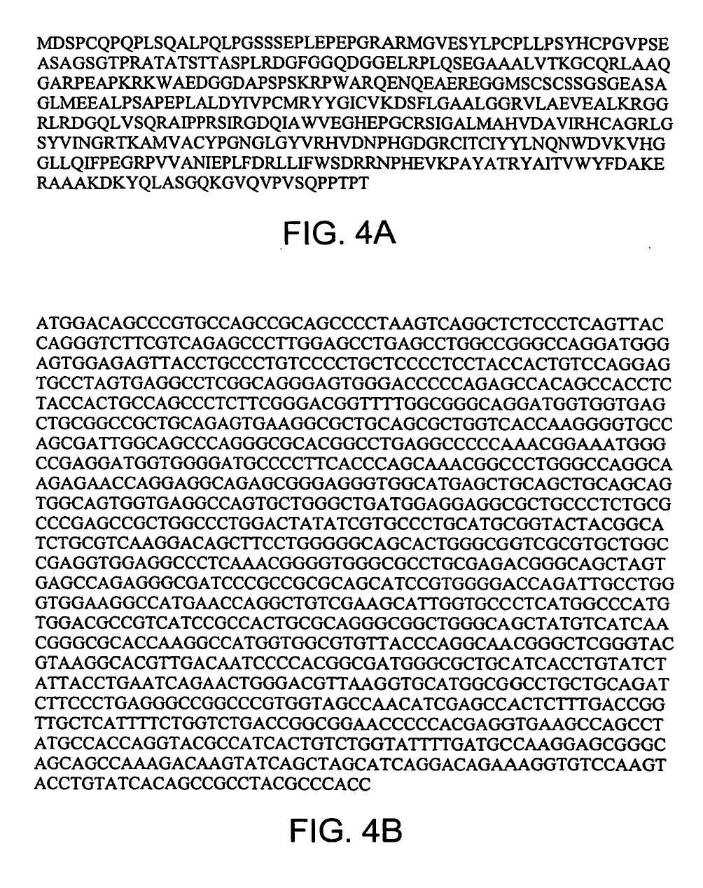

By analyzing and sequencing expressed sequence tag (EST) clones, the nucleotide sequence of cDNA (SEQ ID NO:2; FIG. 4B) encoding EIT-6 was obtained and from this the amino acid sequence (SEQ ID NO:1; FIG. 4A) of EIT-6 was deduced. The EIT-6 cDNA open reading frame is 1,221 base pairs (bp) long; the EIT-6 protein is 407 amino acids long and has a predicted molecular weight of about 43 kDa. The EIT-6 gene was localized to chromosome 19q13.1. Based on nucleotide homology, the EIT-6 was concluded to be a member of a multi-gene family of growth regulatory genes that includes rat SM-20, SM-20 homologues from a variety of species, and a Caenorhabditis elegans gene (egl-9) that was identified as an egg laying defective mutant (FIG. 3B). The proteins encoded by this family of genes have been shown to be dioxygenases that hydroxylate proline residues and have an absolute requirement for dioxygen as a cosubstrate [Epstein et al. (2001) Cell 107:43-54; Bruick et al, Science Express, Oct. 11, 2001]. In contrast to some of the proteins encoded by this family of genes, EIT-6 localizes to the nucleus of the cell. In situ RNA analysis confirmed the findings of SAGE analysis indicating expression of EIT-6 in normal mammary tissue.

Up-regulation of EIT-6 expression by estrogen was not observed in all ER+cells tested and thus it is dependent on other cellular factors. Estrogen-mediated enhancement of EIT-6 expression was shown to be dependent on estrogen receptors. Experiments with transcription and protein synthesis inhibitors indicated that the EIT-6 gene is a direct transcriptional target of estrogen receptors. Transfection experiments indicated that: (a) sequences within a proximal promoter region of about 5.5 kb 5′ of the EIT-6 coding region confer estrogen-responsiveness on the EIT-6 gene; and (b) putative estrogen responsive elements (ERE) in this region have this activity.

Transfection of breast cancer cells with expression constructs containing cDNA encoding EIT-6 indicated that EIT-6 enhances proliferation or survival of both ER+and estrogen non-responsive (ER-) cells. Furthermore, EIT-6 overcame partially, at least, the estrogen dependence of estrogen-dependent cells.

### EIT-6 Nucleic Acid Molecules

The EIT-6 nucleic acid molecules of the invention can be cDNA, genomic DNA, synthetic DNA, or RNA, and can be double-stranded or single-stranded (i.e., either a sense or an antisense strand). They can also be any of a variety of non-naturally occurring nucleic acid analogs known in the art e.g., protein nucleic acids (PNA). Segments of these molecules are also considered within the scope of the invention, and can be produced by, for example, the polymerase chain reaction (PCR) or generated by treatment with one or more restriction endonucleases. A ribonucleic acid (RNA) molecule can be produced by in vitro transcription. Preferably, the nucleic acid molecules encode polypeptides that, regardless of length, are soluble under normal physiological conditions.

The nucleic acid molecules of the invention can contain naturally occurring sequences, or sequences that differ from those that occur naturally, but, due to the degeneracy of the genetic code, encode the same polypeptide (for example, the polypeptides with SEQ ID NO:1). In addition, these nucleic acid molecules are not limited to coding sequences, e.g., they can include some or all of the non-coding sequences that lie upstream or downstream from a coding sequence. The nucleic molecule can, for example, be or comprise a nucleic acid that encodes a polypeptide with SEQ ID NO:1. Thus it can be or comprise the nucleic acid sequence with SEQ ID NO:2. The invention also embodies a nucleic acid molecule which includes a segment of at least 30 (e.g., at least 40, 45, 50, 55, 60, 65, 70, 75, 80, 85, 90, 95, 100, 125, 150, 175, 200, 300, 400, 500, 600, 700, 800, 900, 1000, 1100, 1150, 1200, 1205, 1210, 1215, 1218, 1219, or 1220) nucleotides of SEQ ID NO:2.

The nucleic acid molecules of the invention can be synthesized (for example, by phosphoramidite-based synthesis) or obtained from a biological cell, such as the cell of a mammal. The nucleic acids can be those of a human, non-human primate (e.g., monkey), mouse, rat, guinea pig, cow, sheep, horse, pig, rabbit, dog, or cat. Combinations or modifications of the nucleotides within these types of nucleic acids are also encompassed.

In addition, the nucleic acid molecules of the invention encompass segments that are not found as such in the natural state. Thus, the invention encompasses recombinant nucleic acid molecules (for example, isolated nucleic acid molecules encoding EIT-6) incorporated into a vector (for example, a plasmid or viral vector) or into the genome of a heterologous cell (or the genome of a homologous cell, at a position other than the natural chromosomal location). Recombinant nucleic acid molecules and uses therefor are discussed further below.

Certain nucleic acid molecules of the invention are antisense molecules or are transcribed into antisense molecules. These can be used, for example, to down-regulate translation of EIT-6 mRNA within a cell.

Techniques associated with detection or regulation of genes are well known to skilled artisans. Such techniques can be used to diagnose and/or treat disorders associated with aberrant EIT-6 expression. Nucleic acid molecules of the invention are discussed further below in the context of their use in method of the invention.

Hybridization can be used as a measure of homology between two nucleic acid sequences. An EIT-6-encoding nucleic acid sequence, or a portion thereof, can be used as a hybridization probe according to standard hybridization techniques. The hybridization of a EIT-6 probe to DNA or RNA from a test source (e.g., a mammalian cell) is an indication of the presence of EIT-6 DNA or RNA in the test source. Hybridization conditions are known to those skilled in the art and can be found in Current Protocols in Molecular Biology, John Wiley & Sons, N.Y., 6.3.1-6.3.6, 1991. Moderate hybridization conditions are defined as equivalent to hybridization in 2×sodium chloride/sodium citrate (SSC) at 30° C., followed by a wash in 1×SSC, 0.1% SDS at 50° C. Highly stringent conditions are defined as equivalent to hybridization in 6×sodium chloride/sodium citrate (SSC) at 45° C., followed by a wash in 0.2×SSC, 0.1% SDS at 65° C.

The invention also encompasses: (a) vectors (see below) that contain any of the foregoing EIT-6-related coding sequences and/or their complements (that is, “antisense” sequences); (b) expression vectors that contain any of the foregoing EIT-6-related coding sequences operably linked to any transcriptional/translational regulatory elements (examples of which are given below) necessary to direct expression of the coding sequences; (c) expression vectors encoding, in addition to a EIT-6 polypeptide, a sequence unrelated to EIT-6, such as a reporter, a marker, or a signal peptide fused to EIT-6; and (d) genetically engineered host cells (see below) that contain any of the foregoing expression vectors and thereby express the nucleic acid molecules of the invention.

Recombinant nucleic acid molecules can contain a sequence encoding EIT-6 or EIT-6 having an heterologous signal sequence. The full length EIT-6 polypeptide, or a fragment thereof, may be fused to such heterologous signal sequences or to additional polypeptides, as described below. Similarly, the nucleic acid molecules of the invention can encode the mature form of EIT-6 or a form that includes an exogenous polypeptide that facilitates secretion.

The transcriptional/translational regulatory elements referred to above and further described below include but are not limited to inducible and non-inducible promoters, enhancers, operators and other elements that are known to those skilled in the art and that drive or otherwise regulate gene expression. Such regulatory elements include but are not limited to the cytomegalovirus hCMV immediate early gene, the early or late promoters of SV40 adenovirus, the lac system, the trp system, the TAC system, the TRC system, the major operator and promoter regions of phage A, the control regions of fd coat protein, the promoter for 3-phosphoglycerate kinase, the promoters of acid phosphatase, and the promoters of the yeast α-mating factors.

Similarly, the nucleic acid can form part of a hybrid gene encoding additional polypeptide sequences, for example, a sequence that functions as a marker or reporter. Examples of marker and reporter genes include, without limitation, βlactamase, chloramphenicol acetyltransferase (CAT), adenosine deaminase (ADA), aminoglycoside phosphotransferase (neo^{r}, G418^{r}), dihydrofolate reductase (DHFR), hygromycin-B-phosphotransferase (HPH), thymidine kinase (TK), lacZ (encoding βgalactosidase), xanthine guanine phosphoribosyltransferase (XGPRT), luciferase, green fluorescent protein (GFP), and variants of GFP, e.g., blue fluorescent protein, yellow fluorescent protein, and cyan fluorescent protein. As with many of the standard procedures associated with the practice of the invention, skilled artisans will be aware of additional useful reagents, for example, additional sequences that can serve the function of a marker or reporter. Generally, the hybrid polypeptide will include a first portion and a second portion; the first portion being an EIT-6 polypeptide and the second portion being, for example, the reporter described above or an Ig constant region or part of an Ig constant region, e.g., the CH2 and CH3 domains of IgG2a heavy chain. Other hybrids could include an antigenic tag such hemagglutinin (HA) or a His tag to facilitate purification.

The expression systems that may be used for purposes of the invention include but are not limited to microorganisms such as bacteria (for example, E. coli and B. subtilis) transformed with recombinant bacteriophage DNA, plasmid DNA, or cosmid DNA expression vectors containing the nucleic acid molecules of the invention; yeast (for example, Saccharomyces and Pichia) transformed with recombinant yeast expression vectors containing the nucleic acid molecule of the invention; insect cell systems infected with recombinant virus expression vectors (for example, baculovirus) containing the nucleic acid molecule of the invention; plant cell systems infected with recombinant virus expression vectors (for example, cauliflower mosaic virus (CaMV) or tobacco mosaic virus (TMV)) or transformed with recombinant plasmid expression vectors (for example, Ti plasmid) containing a EIT-6 nucleotide sequence; or mammalian cell systems (for example, COS, CHO, BHK, 293, VERO, HeLa, MDCK, WI38, and NIH 3T3 cells) harboring recombinant expression constructs containing promoters derived from the genome of mammalian cells (for example, the metallothionein promoter) or from mammalian viruses (for example, the adenovirus late promoter and the vaccinia virus 7.5K promoter). Also useful as host cells are primary or secondary cells obtained directly from a mammal and transfected with a plasmid vector or infected with a viral vector.

EIT-6 Polypentides and Polypetptide Fragments

The polypeptides of the invention include EIT-6 and functional fragments of EIT-6. Functional fragments can contain 10, 20, 30, 40, 50, 60, 70, 80, 90, 100, 120, 150, 200, 250, 300, 350, 390, 395, 400, 401, 402, 403, 404, 405, or 406 consecutive amino acid residues of SEQ ID NO:1. The polypeptides embraced by the invention also include fusion proteins that contain either full-length EIT-6 or a functional fragment of it fused to unrelated amino acid sequence. The unrelated sequences can be additional functional domains or signal peptides. The polypeptides can be any of those described above but with not more (i.e., not more than, for example, 50, 45, 40, 35, 30, 25, 20, 17, 14, 12, 10, nine, eight, seven, six, five, four, three, two, or one) conservative substitution(s).

The polypeptides can be purified from natural sources (e.g., tissues or cells such as normal breast epithelial cells or any cell that naturally produces EIT-6 polypeptides). Smaller peptides (less than 100 amino acids long) can also be conveniently synthesized by standard chemical means. In addition, both polypeptides and peptides can be produced by standard in vitro recombinant DNA techniques and in vivo transgenesis, using nucleotide sequences encoding the appropriate polypeptides or peptides. Methods well-known to those skilled in the art can be used to construct expression vectors containing relevant coding sequences and appropriate transcriptional/translational control signals. See, for example, the techniques described in Sambrook et al., Molecular Cloning: A Laboratory Manual (2nd Ed.) [Cold Spring Harbor Laboratory, N.Y., 1989], and Ausubel et al., Current Protocols in Molecular Biology [Green Publishing Associates and Wiley Interscience, N.Y., 1989].

Polypeptides and fragments of the invention also include those described above, but modified for in vivo use by the addition, at the amino- and/or carboxyl-terminal ends, of a blocking agent to facilitate survival of the relevant polypeptide in vivo. This can be useful in those situations in which the peptide termini tend to be degraded by proteases prior to cellular uptake. Such blocking agents can include, without limitation, additional related or unrelated peptide sequences that can be attached to the amino and/or carboxyl terminal residues of the peptide to be administered. This can be done either chemically during the synthesis of the peptide or by recombinant DNA technology by methods familiar to artisans of average skill.

Alternatively, blocking agents such as pyroglutamic acid or other molecules known in the art can be attached to the amino and/or carboxyl terminal residues, or the amino group at the amino terminus or carboxyl group at the carboxyl terminus can be replaced with a different moiety. Likewise, the peptides can be covalently or noncovalently coupled to pharmaceutically acceptable “carrier” proteins prior to administration.

Also of interest are peptidomimetic compounds that are designed based upon the amino acid sequences of the functional peptide fragments. Peptidomimetic compounds are synthetic compounds having a three-dimensional conformation (i.e., a “peptide motif”) that is substantially the same as the three-dimensional conformation of a selected peptide. The peptide motif provides the peptidomimetic compound with the ability to enhance the proliferation of cancer cells (e.g., breast cancer cells) in a manner qualitatively identical to that of the EIT-6 functional fragment from which the peptidomimetic was derived. Peptidomimetic compounds can have additional characteristics that enhance their therapeutic utility, such as increased cell permeability and prolonged biological half-life.

The peptidomimetics typically have a backbone that is partially or completely non-peptide, but with side groups that are identical to the side groups of the amino acid residues that occur in the peptide on which the peptidomimetic is based. Several types of chemical bonds, e.g., ester, thioester, thioamide, retroamide, reduced carbonyl, dimethylene and ketomethylene bonds, are known in the art to be generally useful substitutes for peptide bonds in the construction of protease-resistant peptidomimetics.

EIT-6 Antibodies

The invention features antibodies that bind to EIT-6 polypeptides or fragments of such polypeptides. Such antibodies can be polyclonal antibodies present in the serum or plasma of animals (e.g., mice, rabbits, rats, guinea pigs, sheep, horses, goats, cows, or pigs) which have been immunized with the relevant EIT-6 polypeptide or peptide fragment using methods, and optionally adjuvants, known in the art. Such polyclonal antibodies can be isolated from serum or plasma by methods known in the art. Monoclonal antibodies that bind to the above polypeptides or fragments are also encompassed by the invention. Methods of making and screening monoclonal antibodies are well known in the art.

Once the desired antibody-producing hybridoma has been selected and cloned, the resultant antibody can be produced by a number of methods known in the art. For example, the hybridoma can be cultured in vitro in a suitable medium for a suitable length of time, followed by the recovery of the desired antibody from the supernatant. The length of time and medium are known or can be readily determined.

Additionally, recombinant antibodies specific for EIT-6, such as chimeric and humanized monoclonal antibodies comprising both human and non-human portions, are within the scope of the invention. Such chimeric and humanized monoclonal antibodies can be produced by recombinant DNA techniques known in the art, for example, using methods described in Robinson et al., International Patent Publication PCT/US86/02269; Akira et al., European Patent Application 184,187; Taniguchi, European Patent Application 171,496; Morrison et al., European Patent Application 173,494; Neuberger et al., PCT Application WO 86/01533; Cabilly et al., U.S. Pat. No. 4,816,567; Cabilly et al., European Patent Application 125,023; Better et al. (1988) Science 240:1041-1043; Liu et al. (1987) J. Immunol. 139:3521-3526; Sun et al. (1987) Proc. Natl. Acad. Sci. 84:214-218; Nishimura et al. (1987) Canc. Res. 47:999-1005; Wood et al. (1985) Nature 314:446-449; Shaw et al. (1988) J. Natl. Cancer Inst 80:1553-1559; Morrison, (1985) Science 229:1202-1207; Oi et al. (1986) BioTechniques 4:214; Winter, U.S. Pat. No. 5,225,539; Jones et al. (1986) Nature 321:552-555; Veroeyan et al. (1988) Science 239:1534; and Beidler et al. (1988) J. Immunol. 141:4053-4060.

Also included within the scope of the invention are antibody fragments and derivatives which contain at least the functional portion of the antigen binding domain of an antibody that binds-specifically to EIT-6. Antibody fragments that contain the binding domain of the molecule can be generated by known techniques. For example, such fragments include, but are not limited to: F(ab′)₂ fragments which can be produced by pepsin digestion of antibody molecules; Fab fragments which can be generated by reducing the disulfide bridges of F(ab′)₂ fragments; and Fab fragments which can be generated by treating antibody molecules with papain and a reducing agent. See, e.g., National Institutes of Health, 1 Current Protocols In Immunology, Coligan et al., ed. 2.8, 2.10 (Wiley Interscience, 1991). Antibody fragments also include Fv (e.g., single chain Fv (scFv)) fragments, i.e., antibody products in which there are few or no constant region amino acid residues. An ScFv fragment is a single polypeptide chain that includes both the heavy and light chain variable regions of the antibody from which the ScFv is derived. Such fragments can be produced, for example, as described in U.S. Pat. No. 4,642,334, which is incorporated herein by reference in its entirety.

Methods of Screening for Compounds that Inhibit the Activity of EIT-6

The invention provides in vitro methods for identifying compounds (small molecules or macromolecules) that inhibit an activity of EIT-6. Such an activity can be, for example, the ability of EIT-6 to enhance proliferation or survival of a cell, e.g., a cancer cell such as a breast cancer cell. An alternative activity of EIT-6 that can be inhibited by methods of the invention can be its enzymatic activity, i.e., its prolyl hydroxylase activity.

A method to test for the ability of a test compound to inhibit the ability of EIT-6 to enhance cell proliferation or cell survival can involve, e.g., separately culturing (a) cells expressing EIT-6 and (b) cells that do not substantially express EIT-6 with the test compound. As used herein, a cell that does not “substantially express EIT-6” is a cell (i) in which, using standard methods of detection (e.g., immunohistology, fluorescence flow cytometry, or immunoprecipitation), no EIT-6 protein is detectable or (ii) that expresses at least 2-fold (e.g., at least 2-fold, at least 4-fold, at least 5-fold, at least 10-fold, at least 20-fold, at least 40-fold, at least 50-fold, at least 100-fold, at least 500-fold, at least 1,000-fold, at least 10,000, or even higher-fold) less EIT-6 than cells used in the method that do express EIT-6. A compound that (a) inhibits the proliferation or survival of EIT-6-expressing cells and does not inhibit the proliferation or survival of cells substantially not expressing EIT-6 or (b) inhibits the proliferation or survival of EIT-6-expressing cells more efficiently that it inhibits cells substantially not expressing EIT-6, is a compound that inhibits the activity of EIT-6. Cells that express EIT-6 can be any cells (such as, but not limited to, those disclosed herein) that naturally express EIT-6. Alternatively the cells that express EIT-6 can be recombinant cells transfected or transformed with, and expressing, nucleic acid molecules encoding any of the EIT-6 polypeptides (including functional fragments) disclosed herein. Such cells can be stably or transiently transfected or transformed. The invention also includes methods in which only cells expressing EIT-6 are cultured with the candidate compound. The results of such assays can, for example, be compared to results from prior assays carried out on cells substantially not expressing EIT-6.

The cells substantially lacking expression of an EIT-6 polypeptide and those expressing an EIT-6 polypeptide will preferably, but not necessarily, be of the same histological type. Treatment of the cells with the test compound can be carried out by culturing the cells with a test compound and measuring the level of proliferation or survival of the cells. Alternatively, the cells can be exposed to the test compound for a period of time (e.g., one minute, 10 minutes, 30 minutes, one hour, two hours, four hours, eight hours, 12 hours, 18 hours, 24 hours, two days, three days, 1 week, two weeks, 1 month, 2 months, three months or longer), after which the test compound is removed, and the cells are cultured for an additional period of time (e.g., one minute, 10 minutes, 30 minutes, one hour, two hours, four hours, eight hours, 12 hours, 18 hours, 24 hours, two days, three days, 1 week, two weeks, 1 month, 2 months, three months or longer) and their level of proliferation or survival is measured. Methods of determining relative levels of cell proliferation and/or survival are known in the art, e.g., measurement of [³H]-thymidine incorporation into the DNA of the cells or cell or colony counting using, optionally, a stain or a dye that is excluded by viable cells (e.g., trypan blue or eosin) or a dye that stains all cells, e.g., crystal violet.

In methods to test for the ability of a test compound to inhibit the enzymatic activity of EIT-6, it is possible to add the test compound to a direct assay that measures the ability of EIT-6 to hydroxylate proline residues in appropriate naturally occurring or synthetic polypeptides or small peptide fragments [Epstein et al. (2001)]. In such assays the hydroxylated product can be detected by any of a number of methods known in the art, e.g., high pressure liquid chromatography and/or mass spectroscopy. Suitable polypeptides include collagen, hypoxia inducible factor (HIF)-1, or HIF-2, or proline-containing peptide fragments of these [Dowell et al. (1992) J. Med. Chem. 35:800-804; Epstein et al. (2001)]. These polypeptide substrates can be naturally-occurring or non-naturally-occurring. They can be extracted from natural sources (e.g., cells, tissues, or whole organisms). Alternatively, they can be chemically synthesized, produced by recombinant means or by any of the methods described above for the EIT-6 polypeptides of the invention. Proline-containing amino acid motifs that have been indicated to be conserved sites for proline hydroxylation by prolyl hydroxylases include “LXXLAP” (SEQ ID NO:31) [Epstein et al. (2001)] or “XPG” [Dowell et al. (1992)] in both of which the X residues can be any amino acid. Repeat sequences of the “PPG” motif in collagen have also been shown to be targets for proline hydroxylation by the a₁ and a₂ mammalian prolyl-4-hydroxylases that modify collagen [Jaakola et al. (2001) Science 292:468-472].

The activity of EIT-6 can be also be assayed indirectly by measuring the production of succinic acid produced by decarboxylation of the cosubstrate 2-oxoglutaric acid in the presence of a proline-containing polypeptide substrate, e.g., poly-PGP [Cunliffe et al. (1986) Biochem. J. 240:617-619; Epstein et al. (2001)]. For example, radiolabeled 2-oxo-[5-¹⁴C]glutaric acid can be used as the cosubstrate which, after the reaction, can be separated from the radiolabeled product [1-¹⁴C]succinic acid by ion-exchange or HPLC chromatography [Cunliffe et al. (1986); Epstein et al. (2001)].

The enzyme reactions can be carried out in vivo (e.g., in live cells) but are preferably carried out in vitro using EIT-6 (a) in cell or tissue extracts, (b) isolated from cells or tissues, or (c) synthesized by any of the methods described herein. The assays can also employ any of the forms of EIT-6 disclosed herein, provided only that the relevant form retains the described enzymatic activity. Enzyme reactions are performed under the conditions previously described [see, for example, Cunliffe et al. (1986); Epstein et al. (2001); Jaakola et al. (2001)] or modifications of these conditions that would be obvious to one skilled in the art.

A candidate compound that inhibits the proliferation or survival of cells expressing EIT-6 by more than 20% (e.g., more than 20%, more than 30%, more than 40%, more than 50%, more than 60%, more than 70%, more than 80%, more than 90%, more than 95%, more than 98%, more than 99%, or 100%) relative to the proliferation of cells substantially not expressing EIT-6 is a compound that inhibits the activity of EIT-6 and is one that could be useful as, for example, a cancer therapeutic agent. In addition, a compound that inhibits the enzymatic activity of EIT-6 by more than 20% (e.g., more than 20%, more than 30%, more than 40%, more than 50%, more than 60%, more than 70%, more than 80%, more than 90%, more than 95%, more than 98%, more than 99%, or 100%), in that it is a compound that inhibits the activity of EIT-6, is one that could be also be useful as, for example, a cancer therapeutic agent.

The invention also relates to the use of any of the forms of EIT-6 described herein to screen for compounds that can interact with EIT-6 and potentially thereby inhibit its activity. One of skill in the art would know how to use standard molecular modeling or other techniques to identify small molecules that would bind to appropriate sites (e.g., substrate-binding or allosteric sites) on EIT-6. One such example is provided in Broughton (1997) Curr. Opin. Chem. Biol. 1, 392-398.

Methods of Inhibiting an Activity of EIT-6

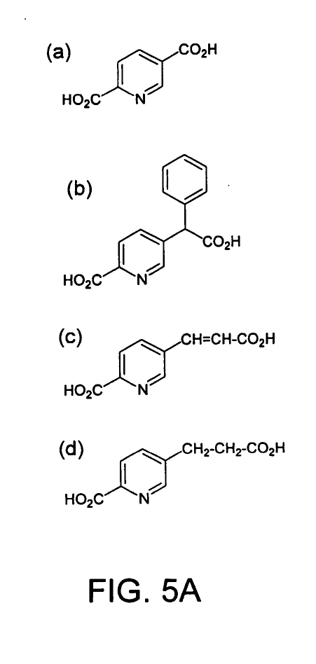

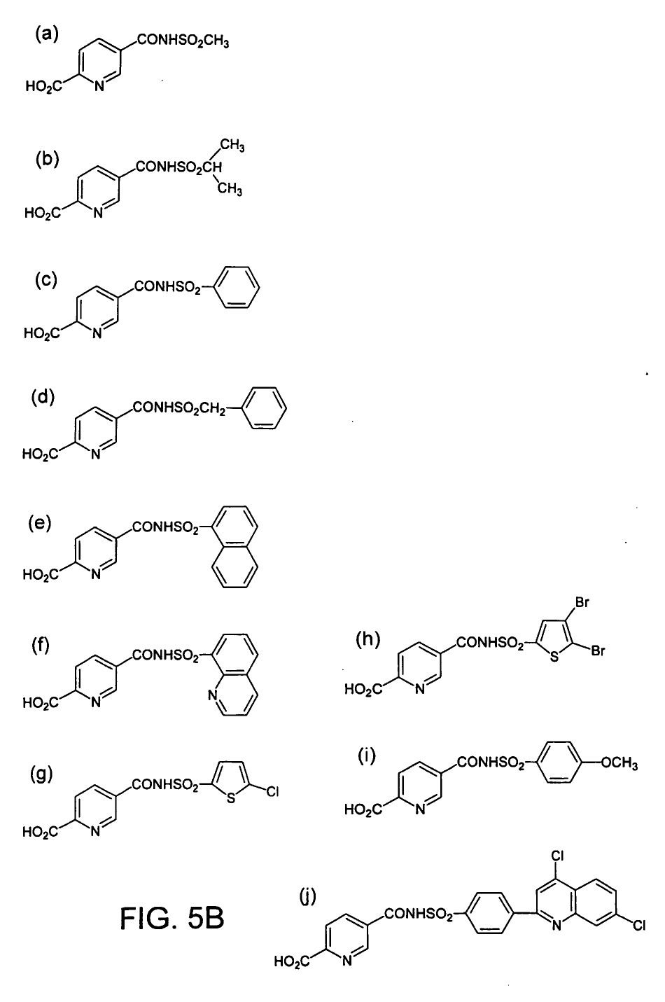

The invention features a method of inhibiting an activity of EIT-6. The method involves contacting EIT-6 with a compound identified (as described above) as one that inhibits the activity of EIT-6. Alternatively, EIT-6 can be contacted with any of a number of compounds previously identified as having the ability to inhibit prolyl hydroxylase enzymes. Such compounds include analogs of the prolyl hydroxylase cosubstrate, 2-oxoglutarate. Inhibitory analogs of 2-oxoglutarate, include N-oxalylglycine and N-oxalyl-2S-alanine (but not the enantiomer N-oxalyl-2R-alanine). The dimethyl ester of N-oxalylglycine (dimethyl-oxalylglycine) is particularly useful in that it penetrates cells more efficiently than N-oxalylglyicine [Epstein et al. (2001); Jaakola et al. (2001) Science 292:468-472; Cunliffe et al. (1992) J. Med. Chem. 35:2652-2658]. It is anticipated that mono- and dialkyl esters with relatively short alkyl groups (e.g., methyl, ethyl, propyl, or butyl) can be similarly useful. In addition, pyridine-2,5-dicarboxylic acid (“P2,5D”) and derivatives thereof can be useful inhibitors of EIT-6 [Dowell. et al. (1986) J. Med. Chem. 35:800-804]; it was found that compounds having any of a variety of groups between the pyridine ring of P2,5D and the carboxylic group in the 5 position (e.g., the compounds shown in FIG. 5A) were able to inhibit proline hydroxylase activity almost as efficiently as P2,5D. It was also found that compounds (FIG. 5B) resulting from the replacement of the carboxyl group at the 5 position of P2,5D with a variety of acyl sulfonamides were even more potent inhibitors than P2,5D [Dowell et al. (1986)]. Thus, the invention includes, but is not limited to, the use of all the above-described compounds and analogs in the methods of inhibiting the enzymic activity of EIT-6.

While not necessarily the case, the EIT-6 inhibited by the methods of the invention will preferably be in a cell (e.g., a cancer cell such as a breast cancer, lung cancer, colon cancer, pancreatic cancer, renal cancer, stomach cancer, liver cancer, bone cancer, hematological cancer (e.g., leukemia or lymphoma), neural tissue cancer, melanoma, ovarian cancer, testicular cancer, prostate cancer, cervical cancer, vaginal cancer, or bladder cancer cell). The cell can be an estrogen responsive cell (ER+) or an estrogen non-responsive (ER−) cell.

The methods can be performed in vitro, in vivo, or ex vivo. In vitro application of compounds that inhibit EIT-6 can be useful, for example, in basic scientific studies of tumor cell biology, e.g., studies on the mechanism of action of EIT-6. In addition, compounds that inhibit EIT-6 can be used as “positive controls” in methods to identify additional compounds with inhibitory activity (see above). In such in vitro methods, the EIT-6, or cells expressing EIT-6, can be incubated for various times with the inhibitory compound(s) at a variety of concentrations. Other incubation conditions known to those in art (e.g., temperature, EIT-6 concentration, or cell concentration) can also be varied. Inhibition of EIT-6 activity can be tested by methods such as those disclosed herein.

The methods of the invention will preferably be in vivo.

Compounds that inhibit EIT-6 are generally useful as cancer cell (e.g., breast cancer cell) proliferation-inhibiting or survival-inhibiting therapeutics. They can be administered to mammalian subjects (e.g., human breast cancer patients) alone or in conjunction with other drugs and/or radiotherapy. As used herein, a compound that is “therapeutic” is a compound that causes a complete abolishment of the symptoms of a disease or a decrease in the severity of the symptoms of the disease. “Prevention” should mean that symptoms of the disease (e.g., cancer) are essentially absent.

These methods of the invention can be applied to a wide range of species, e.g., humans, non-human primates, horses, cattle, pigs, sheep, goats, dogs, cats, rabbits, guinea pigs, hamsters, rats, and mice.

In Vivo Approaches

In one in vivo approach, a compound that inhibits the activity of EIT-6 (see above) is administered to a subject. Generally, the compounds of the invention will be suspended in a pharmaceutically-acceptable carrier (e.g., physiological saline) and administered orally, intravenously, subcutaneously, intradermally, intramuscularly, intrathecally, intraperitoneally, intrarectally, intravaginally, intranasally, intragastrically, intratracheally, or intrapulmonarily. They can also be delivered directly to tumor cells, e.g., to a tumor or a tumor bed following surgical excision of the tumor, in order to kill any remaining tumor cells. The dosage required depends on the choice of the route of administration; the nature of the formulation; the nature of the patient's illness; the subject's size, weight, surface area, age, and sex; other drugs being administered; and the judgment of the attending physician. Suitable dosages are in the range of 0.001 mg/kg-100 mg/kg. Wide variations in the needed dosage are to be expected in view of the variety of compounds available and the differing efficiencies of various routes of administration. For example, oral administration would be expected to require higher dosages than administration by intravenous injection. Variations in these dosage levels can be adjusted using standard empirical routines for optimization as is well understood in the art. Administrations can be single or multiple (e.g., 2-, 3-, 4-, 6-, 8-, 10-, 20-, 50-,100-, 150-, or more fold). Encapsulation of the polypeptide in a suitable delivery vehicle (e.g., polymeric microparticles or implantable devices) may increase the efficiency of delivery, particularly for oral delivery.

Alternatively, where an inhibitory compound is a polypeptide, a polynucleotide containing a nucleic acid sequence encoding the polypeptide can be delivered to appropriate cells in a mammal. Expression of the coding sequence can be directed to any cell in the body of the subject. However, expression will preferably be directed to cells in the vicinity of the tumor cells whose proliferation it is desired to inhibit. Expression of the coding sequence can be directed to the tumor cells themselves. This can be achieved by, for example, the use of polymeric, biodegradable microparticle or microcapsule delivery devices known in the art.

Another way to achieve uptake of the nucleic acid is using liposomes, prepared by standard methods. The vectors can be incorporated alone into these delivery vehicles or co-incorporated with tissue-specific or tumor-specific antibodies. Alternatively, one can prepare a molecular conjugate composed of a plasmid or other vector attached to poly-L-lysine by electrostatic or covalent forces. Poly-L-lysine binds to a ligand that can bind to a receptor on target cells [Cristiano et al. (1995), J. Mol. Med. 73:479]. Alternatively, tissue specific targeting can be achieved by the use of tissue-specific transcriptional regulatory elements (TRE) which are known in the art. Delivery of “naked DNA” (i.e., without a delivery vehicle) to an intramuscular, intradermal, or subcutaneous site is another means to achieve in vivo expression.

In the relevant polynucleotides (e.g., expression vectors), the nucleic acid sequence encoding the polypeptide of interest with an initiator methionine and optionally a targeting sequence is operatively linked to a promoter or enhancer-promoter combination. Short amino acid sequences can act as signals to direct proteins to specific intracellular compartments. Such signal sequences are described in detail in U.S. Pat. No. 5,827,516, incorporated herein by reference in its entirety.

Enhancers provide expression specificity in terms of time, location, and level. Unlike a promoter, an enhancer can function when located at variable distances from the transcription initiation site, provided a promoter is present. An enhancer can also be located downstream of the transcription initiation site. To bring a coding sequence under the control of a promoter, it is necessary to position the translation initiation site of the translational reading frame of the peptide or polypeptide between one and about fifty nucleotides downstream (3′) of the promoter. The coding sequence of the expression vector is operatively linked to a transcription terminating region.

Suitable expression vectors include plasmids and viral vectors such as herpes viruses, retroviruses, vaccinia viruses, attenuated vaccinia viruses, canary pox viruses, adenoviruses and adeno-associated viruses, among others.

Polynucleotides can be administered in a pharmaceutically acceptable carrier. Pharmaceutically acceptable carriers are biologically compatible vehicles that are suitable for administration to a human, e.g., physiological saline or liposomes. A therapeutically effective amount is an amount of the polynucleotide that is capable of producing a medically desirable result (e.g., decreased proliferation of cancer cells) in a treated animal. As is well known in the medical arts, the dosage for any one patient depends upon many factors, including the patient's size, body surface area, age, the particular compound to be administered, sex, time and route of administration, general health, and other drugs being administered concurrently. Dosages will vary, but a preferred dosage for administration of polynucleotide is from approximately 10⁶ to approximately 10¹² copies of the polynucleotide molecule. This dose can be repeatedly administered, as needed. Routes of administration can be any of those listed above.

### Ex Vivo Approaches

An ex vivo strategy can involve transfecting or transducing cells obtained from the subject with a polynucleotide encoding a polypeptide that inhibits EIT-6. The transfected or transduced cells are then returned to the subject. The cells can be any of a wide range of types including, without limitation, hemopoietic cells (e.g., bone marrow cells, macrophages, monocytes, dendritic cells, T cells, or B cells), fibroblasts, epithelial cells, endothelial cells, keratinocytes, or muscle cells. Such cells act as a source of the inhibitory polypeptide for as long as they survive in the subject. Alternatively, tumor cells, preferably obtained from the subject but potentially from an individual other than the subject, can be transfected or transformed by a vector encoding the inhibitory polypeptide. The tumor cells, preferably treated with an agent (e.g., ionizing irradiation) that ablates their proliferative capacity, are then introduced into the patient, where they secrete the polypeptide.

The ex vivo methods include the steps of harvesting cells from a subject, culturing the cells, transducing them with an expression vector, and maintaining the cells under conditions suitable for expression of the polypeptide that inhibits the activity of EIT-6 These methods are known in the art of molecular biology. The transduction step is accomplished by any standard means used for ex vivo gene therapy, including calcium phosphate, lipofection, electroporation, viral infection, and biolistic gene transfer. Alternatively, liposomes or polymeric microparticles can be used. Cells that have been successfully transduced can then be selected, for example, for expression of the coding sequence or of a drug resistance gene. The cells may then be lethally irradiated (if desired) and injected or implanted into the patient.

Methods of Inhibiting Expression of EIT-6 in a Cell

Also included in the invention are methods of inhibiting expression in cells. The method involves introducing into a cell (a) an antisense oligonucleotide or (b) a nucleic acid comprising a transcriptional regulatory element (TRE) operably linked to a nucleic sequence that is transcribed in the cell into an antisense RNA. The antisense oligonucleotide and the antisense RNA hybridize of an EIT-6 transcript and have the effect in the cell of inhibiting expression of EIT-6 in the cell. Inhibiting EIT-6 expression in the cell can inhibit proliferation or survival of the cell. The method can thus be useful in inhibiting proliferation of a cancer cell and can be applied to the therapy of cancer.

Antisense compounds are generally used to interfere with protein expression either by, for example, interfering directly with translation of a target mRNA molecule, by RNAse-H-mediated degradation of the target mRNA, by interference with 5′ capping of mRNA, by prevention of translation factor binding to the target mRNA by masking of the 5′ cap, or by inhibiting of mRNA polyadenylation. The interference with protein expression arises from the hybridization of the antisense compound with its target mRNA. A specific targeting site on a target mRNA of interest for interaction with a antisense compound is chosen. Thus, for example, for modulation of polyadenylation a preferred target site on an mRNA target is a polyadenylation signal or a polyadenylation site. For diminishing mRNA stability or degradation, destabilizing sequence are preferred target sites. Once one or more target sites have been identified, oligonucleotides are chosen which are sufficiently complementary to the target site (i.e., hybridize sufficiently well under physiological conditions and with sufficient specificity) to give the desired effect.

With respect to this invention, the term “oligonucleotide” refers to an oligomer or polymer of RNA, DNA, or a mimetic of either. The term includes oligonucleotides composed of naturally-occurring nucleobases, sugars, and covalent internucleoside (backbone) linkages. The normal linkage or backbone of RNA and DNA is a 3′ to 5′ phosphodiester bond. The term also refers however to oligonucleotides composed entirely of, or having portions containing, non-naturally occurring components which function in a similar manner to the oligonucleotides containing only naturally-occurring components. Such modified substituted oligonucleotides are often preferred over native forms because of desirable properties such as, for example, enhanced cellular uptake, enhanced affinity for target sequence, and increased stability in the presence of nucleases. In the mimetics, the core base (pyrimidine or purine) structure is generally preserved but (1) the sugars are either modified or replaced with other components and/or (2) the inter-nucleobase linkages are modified. One class of nucleic acid mimetic that has proven to be very useful is referred to as protein nucleic acid (PNA). In PNA molecules the sugar backbone is replaced with an amide-containing backbone, in particular an aminoethylglycine backbone. The bases are retained and are bound directly to the aza nitrogen atoms of the amide portion of the backbone. PNA and other mimetics useful in the instant invention are described in detail in U.S. Pat. No. 6,210,289.

The antisense oligomers to be used in the methods of the invention generally comprise about 8 to about 100 (e.g., about 14 to about 80 or about 14 to about 35) nucleobases (or nucleosides where the nucleobases are naturally occurring).

The antisense oligonucleotides can themselves be introduced into a cell or an expression vector containing a nucleic sequence (operably linked to a TRE) encoding the antisense oligonucleotide can be introduced into the cell. In the latter case, the oligonucleotide produced by the expression vector is an RNA oligonucleotide and the RNA oligonucleotide will be composed entirely of naturally occurring components.

The methods of the invention can be in vitro or in vivo. In vitro applications of the methods can be useful, for example, in basic scientific studies on cell proliferation or cell survival. In such in vitro methods, appropriate cells (e.g., those expressing EIT-6), can be incubated for various lengths of time with (a) the antisense oligonucleotides or (b) expression vectors containing nucleic acid sequences encoding the antisense oligonucleotides at a variety of concentrations. Other incubation conditions known to those in art (e.g., temperature or cell concentration) can also be varied. Inhibition of EIT-6 expression can be tested by methods known to those in the art, e.g., methods such as those disclosed herein. However, the methods of the invention will preferably be in vivo.

The antisense methods are generally useful for cancer cell (e.g., breast cancer cell) proliferation-inhibiting or survival-inhibiting therapy. They can be administered to mammalian subjects (e.g., human breast cancer patients) alone or in conjunction with other drugs and/or radiotherapy. Doses, formulations, routes of administration, vectors, and targeting are as described for in vivo approaches to inhibiting the activity of EIT-6. Naturally, the antisense oligonucleotides and expression vectors containing nucleic acid sequences encoding the antisense oligonucleotides will preferably be targeted to cells whose proliferation it is desired to inhibit.

The antisense methods of the invention can be applied to a wide range of species, e.g., humans, non-human primates, horses, cattle, pigs, sheep, goats, dogs, cats, rabbits, guinea pigs, hamsters, rats, and mice.

Double-stranded interfering RNA (RNAi) homologous to EIT-6 DNA can also be used to reduce expression of EIT-6 in a cell. See, e.g., Fire et al. (1998) Nature 391:806-811, Romano and Masino (1992) Mol. Microbiol. 6:3343-3353, Cogoni et al. (1996) EMBO J. 15:3153-3163, Cogoni and Masino (1999) Nature 399:166-169, Misquitta and Paterson (1999) Proc. Natl. Acad. Sci. USA 96:1451-1456, and Kennerdell and Carthew (1998) Cell 95:1017-1026.

The sense and anti-sense RNA strands of RNAi can be individually constructed using chemical synthesis and enzymatic ligation reactions using procedures known in the art. For example, each strand can be chemically synthesized using naturally occurring nucleotides or variously modified nucleotides designed to increase the biological stability of the molecule or to increase the physical stability of the duplex formed between the sense and anti-sense strands, e.g., phosphorothioate derivatives and acridine substituted nucleotides. The sense or anti-sense strand can also be produced biologically using an expression vector into which a target EIT-6 sequence (full-length or a fragment) has been subcloned in a sense or anti-sense orientation. The sense and anti-sense RNA strands can be annealed in vitro before delivery of the dsRNA to any of cancer cells disclosed herein. Alternatively, annealing can occur in vivo after the sense and anti-sense strands are sequentially delivered to the cancer cells.

Double-stranded RNA interference can also be achieved by introducing into cancer cells a polynucleotide from which sense and anti-sense RNAs can be transcribed under the direction of separate promoters, or a single RNA molecule containing both sense and anti-sense sequences can be transcribed under the direction of a single promoter.

One of skill in the art will appreciate that RNAi methods can be, as for the antisense methods described above, in vitro and in vivo. Moreover, methods and conditions of delivery and species to which the RNAi methods can be applied are essentially the same as those for antisense oligonucleotides.

The following examples are meant to illustrate, not limit, the invention.

### EXAMPLE 1.

### Serial Analysis of Gene Expression (SAGE) in Breast Cancer Cells After Treatment with Estrogen or Tamoxifen

In order to identify genes involved in the response of breast cancer cells to estrogen and tamoxifen, SAGE libraries were generated from an estrogen- dependent human breast cancer cell line (ZR75-1; obtained from the American type Culture Collection (ATTC), Manassas, VA) prior to and following estrogen or tamoxifen treatment. SAGE facilitated the determination of the absolute abundance of thousands of different mRNAs simultaneously in a comprehensive and unbiased way and to detect even slight differences in expression levels between samples [Velculescu et al. (1995) Science 270: 484-487; Polyak et al. (1997) Nature 389:300-305; Lal et al. (1999) Cancer Res. 59:5403-5607; Velculescu et al. (1999) Nature Genetics 23:387-388]. ZR75-1 cells cultured in the absence of steroid hormones (i.e., in phenol red-free culture medium containing charcoal-treated fetal bovine serum (FBS) (5%)) for seven days were split into three aliquots which were then cultured in fresh steroid-free medium containing no additive, 10 nM estradiol, or 10 μM 4-hydroxy-tamoxifen. Cells were harvested from the cultures after sixteen hours and response to the hormone treatment was confirmed by FACS analysis of cell cycle progression and by Northern blot analysis to test for the expression of known estrogen target genes. ZR75-1 cells grown without estrogen arrested in G1 and G2 phases of the cell cycle, while addition of estrogen, and to a lesser degree tamoxifen, stimulated rapid S-phase entry.

SAGE libraries from untreated, estrogen-treated and tamoxifen-treated ZR75-1 cells were generated using a modified micro-SAGE protocol [Porter et al. (2001) Cancer Res. 61: 5697-5702]. From the three SAGE libraries 140,638 tags were obtained, approximately 45,000 from each library. Hierarchical clustering was applied to data using the Auster program developed by Eisen et al. [(1998) Proc. Natl. Acad. Sci. USA. 95: 14863-14868]. Data was log-transformed and filtered for at least 1 observations abs Val 5 and Maxval-Minval >5. Using these settings 2,818 genes (out of 16,808 total) were included in the analysis that led to the identification of several estrogen- and/or tamoxifen-induced transcripts. There were 61 tags (33 up-regulated and 22 down-regulated) that showed at least a two-fold difference in number (p<0.001) between the estrogen treated and control libraries, while 15 tags (9 up-regulated and 6 down-regulated) showed at least a two-fold difference (p<0.001) between the tamoxifen-treated and control libraries. In addition, 22 tags were found to be significantly elevated in the estrogen treated cells when compared to the tamoxifen-treated cells, while the levels of 24 tags were significantly elevated in the tamoxifen treated library compared to the estrogen treated library. Linking the UniGene database to the SAGE data identified the cDNAs corresponding to the SAGE tags in most of the cases (Table 1). Genes were named according to their relative abundance in the three SAGE libraries: EIT (Estrogen Induced Tag)-induced by estrogen; TIT (Tamoxifen Induced Tag)-induced by tamoxifen; and DET (Differently Expressed Tag)-differently expressed following estrogen or tamoxifen treatment.

TABLE 1

Estrogen and Tamoxifen Regulated Genes

SEQ
ID

| | | | | | | | |
|---|---|---|---|---|---|---|---|
| Name | SAGE tag | NO: | C | E | T | Genbank # | Function |

| | | | | | | | |
|---|---|---|---|---|---|---|---|
| EIT-1 | GCGGTGACAG | 11 | 1 | 14 | 8 | NA | No reliable database match |
| EIT-2 | TACGAAGTTC | 12 | 1 | 14 | 9 | AF077039 | TIM17B |
| EIT-3 | AATGAGTTTG | 13 | 2 | 19 | 13 | AF201940 | DC6 mRNA |
| EIT-4 | GTCTTAACTC | 14 | 2 | 18 | 9 | BC001046 | Yeast Dim 1 homologue |
| EIT-5 | GTGGCATCAC | 15 | 5 | 27 | 18 | AB043104 | Nop10p snRNP |
| EIT-6 | GGTGTGGAAG | 16 | 5 | 26 | 11 | AY040565 | SM-20 homologue |
| EIT-10 | GAAATACAGT | 17 | 112 | 451 | 315 | X05344 | Cathepsin D ECM protease |
| TIT-1 | CAACGAAACC | 18 | 0 | 4 | 14 | NA | No reliable database match |
| TIT-2 | GCGTGCTCTC | 19 | 0 | 3 | 11 | NA | No reliable database match |
| TIT-3 | GGGGGCCCCG | 20 | 8 | 28 | 32 | AF004876 | Yeast Yiflp homologue |
| TIT-4 | GGGGCCCCCT | 21 | 7 | 27 | 28 | Z96932 | Sjogren's syndrome antigen |
| TIT-5 | CCACCCCGAA | 22 | 9 | 19 | 34 | BC000916 | BI-I bax antagonist |
| DET-1 | TCTCTGCAAA | 23 | 4 | 15 | 1 | BC000890 | Hypothetical protein FLJ20640 |
| DET-2 | TGGATCCTCG | 24 | 3 | 13 | 1 | BC001239 | Hypothetical protein FLJ10479 |
| DET-13 | ATGAAAACTC | 25 | 7 | 1 | 11 | AA524901 | ESTs no homology |
| DET-14 | AGCCACCGTG | 26 | 4 | 1 | 12 | NA | No reliable database match |
| DET-15 | CCCCCGCGGA | 27 | 11 | 1 | 12 | AF130366 | USF2 transcription factor |
| DET-16 | TTTGCGGTCC | 28 | 7 | 0 | 11 | BC002972 | TSC-22-like protein |
| DET-17 | GCTGGGGACT | 29 | 0 | 1 | 10 | NM_001055 | SULT 1A sulfotransferase |

Since the abundance of a given SAGE tag obtained in the analysis reflects the absolute abundance of the corresponding mRNA, data obtained from different experiments performed in different laboratories are directly comparable [Velculescu et al. (1995)]. Therefore, the data obtained in above-described SAGE analyses and data of others from SAGE libraries generated from untreated or estrogen treated MCF-7 cells by others [Charpentier et al. (2000) Cancer Res. 60:5977-5983] were analyzed together using a clustering algorithm to delineate similarities and differences between the effects of estrogen on two different breast cancer cell lines. The two breast cancer cell lines (ZR75-1 and MCF-7) had distinct gene expression patterns and demonstrated a discrete transcriptional response to estrogen treatment [Perou et al. (2000) Nature 406:747-752]. For example, among known estrogen target genes, cathepsin D was induced by estrogen in both cells lines, whereas pS2 and cyclin D1 were induced only in MCF-7 cells. All three SAGE libraries derived from the same cell line were more similar to each other, even after estrogen treatment, than to the other cell line. Interestingly, untreated and estrogen-treated MCF-7 cells were highly similar to each other and untreated tamoxifen-treated ZR75-1 cells were somewhat similar to each other and distinct from estrogen-treated cells. These findings indicate that estrogen exerts an effect that depends on the cellular context and, overall, there are relatively few genes significantly affected by estrogen or tamoxifen treatment in these breast cancer cell lines.

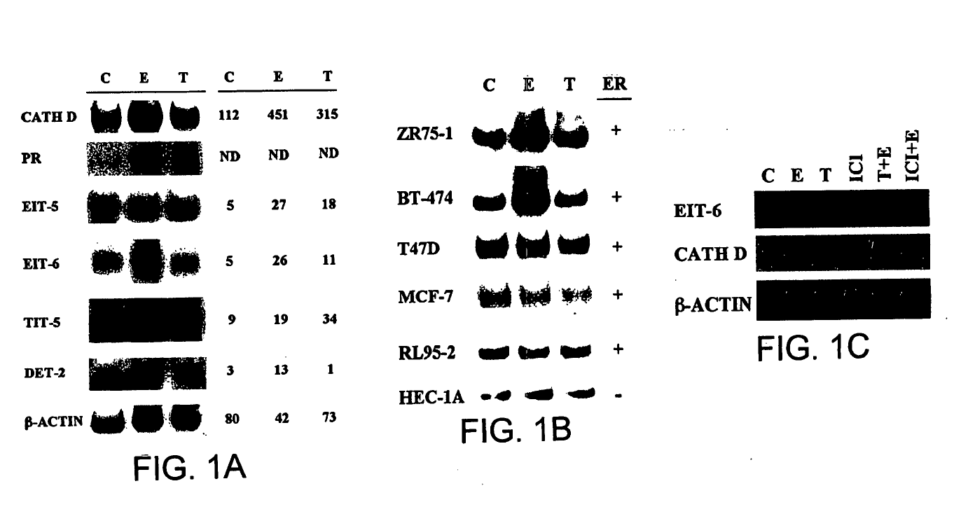

To confirm the result of the SAGE experiment, probes corresponding to some of the cDNA clones were generated and the induction of the relevant genes was confirmed by Northern blot analysis (FIG. 1A) performed as previously described [Polyak et al. (1997)]. Of the twenty estrogen- or tamoxifen-induced genes (Table 1), only one (EIT-10;cathepsin D) had previously been implicated as a target of ER-transcriptional activation and three had not previously been described at all. In most cases the sequences of the relevant genes provided important clues as to their potential functions (Table 1). Several of the genes are predicted to be involved in the regulation of cell proliferation and/or survival. EIT-2 is a protein translocase involved in importing nuclear encoded proteins into mitochondria [Bauer et al. (1999) J. Mol. Biol. 289:69-82]; EIT-4 is a human homologue of the yeast Dim1p gene essential for mitosis [Berry et al. (1997) J. Cell Biol. 137:1337-1354]; EIT-6 is homologous to a rat immediate-early gene SM-20 [Wax et al. (1994) J. Biol. Chem. 269:13041-13047]; TIT-5 is an anti-apoptotic member of the bcl-2 family [Xu et al. (1998) Mol. Cell 1:337-346]; and DET-15 and DET-16 are both putative transcription factors with anti-proliferative activity [Ismail et al. (1999) Oncogene 18:5582-5591; Nakashiro et al. (1998) Cancer Res. 58:549-555; Shibanuma et al. (1992) J. Biol. Chem. 267:10219-10214]. Although several ESTs identified by the analysis have no homology to known genes, their expression pattern in other SAGE libraries suggests that they also might play a role in cell proliferation and/or estrogen mediated responses [Lal et al. (1999) Cancer Res. 59:5403-5407]. For example, the TIT-3 and TIT-1 genes appear to be elevated in ER+(estrogen responsive) DCIS (Ductal Carcinoma In Situ) compared to corresponding normal mammary epithelium and are expressed at much lower levels in other cell types [Porter et al. (2001)]. Interestingly, one of the tamoxifen induced genes, SULT1A phenol sulfotransferase, is an enzyme involved in the metabolism of environmental carcinogens and steroid hormones including estrogen and tamoxifen [Weinshilboum et al. (1997) Faseb J. 11:3-14]. Polymorphism in SULT1A1 was recently shown to influence the age-of onset and the risk of breast cancer [Seth et al. (2000) Cancer Res. 60:6859-6863; Zheng et al. (2001) Cancer Epidemiol Biomarkers Prev. 10:89-94].

### EXAMPLE 2.

### Regulation of EIT-6 Expression by Estrogen

Although the above-described SAGE analysis led to the isolation of several novel estrogen and/or tamoxifen regulated genes, it provided no information as to which of these genes could be key mediators of the cellular response initiated by estrogen and/or tamoxifen. However, one of the identified estrogen induced genes, EIT-6, was particularly interesting in that there was a relatively high abundance of its SAGE tag in SAGE libraries prepared from hormone responsive tissues (normal and cancerous mammary, and ovarian and prostate epithelium) [Lal et al. (1999) Cancer Res. 59:5403-5407]. A full-length (2,071 bp) human EIT-6 cDNA was obtained by analyzing and sequencing EST clones. The human EIT-6 cDNA contains an open reading frame (ORF) of 1,221 bp encoding a protein of 407 amino acids (˜43,000 Daltons), which was confirmed by in vitro transcription/translation experiments. The amino acid sequence (SEQ ID NO:1) of full-length human EIT-6 is shown in FIG. 4A and the nucleotide sequence of cDNA encoding the full-length human EIT-6 is shown in FIG. 4B. By FISH (fluorescence in situ hybridization) analysis the EIT-6 gene was localized to the chromosomal region 19q13. 1, a region not previously implicated in the etiology of breast cancer. The in vivo abundance of the EIT-6 mRNA and its expression at the cellular level was determined by mRNA in situ hybridization of normal human breast tissue, which was carried out using ³³P-labeled sense or anti-sense EIT-6 ribo-probes by a previously described method [Rosen et al. (1990) Mol. Cell 4:611-617]. EIT-6 hybridization signal showed fairly even intensity throughout the mammary epithelium, while no significant signal was detected in stromal cells.

To determine how generally EIT-6 is regulated by estrogen, Northern blot analysis was performed with RNA from various ER+breast and endometrial cancer cell lines (FIG. 1B). All the cell lines were obtained from the ATCC. In addition to being induced in ZR75-1 cells (˜5 fold induction), the cell line used for the generation of the SAGE libraries, EIT-6 was found to be induced in BT-474 cells (˜10 fold induction), but not in other estrogen responsive cell lines analyzed. This finding is not unexpected, since many estrogen targets are induced in a cell type-specific manner.

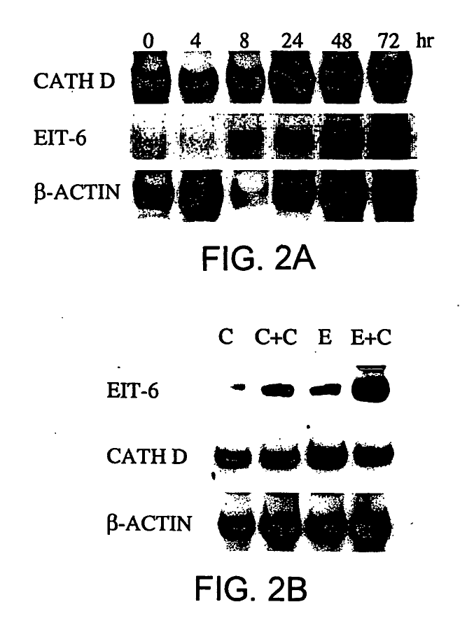

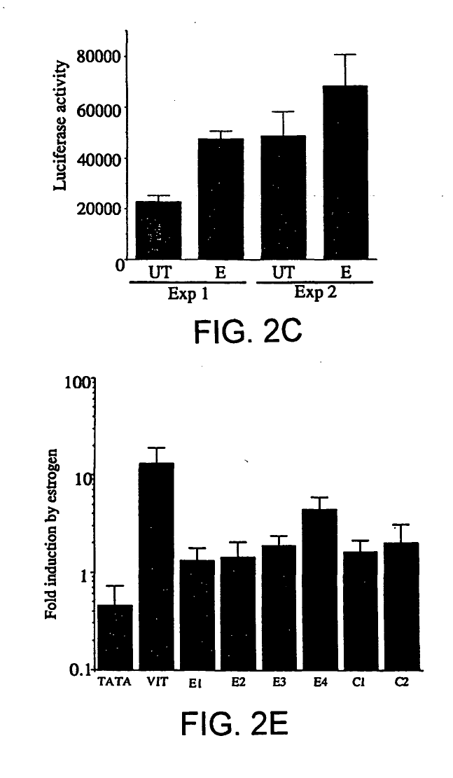

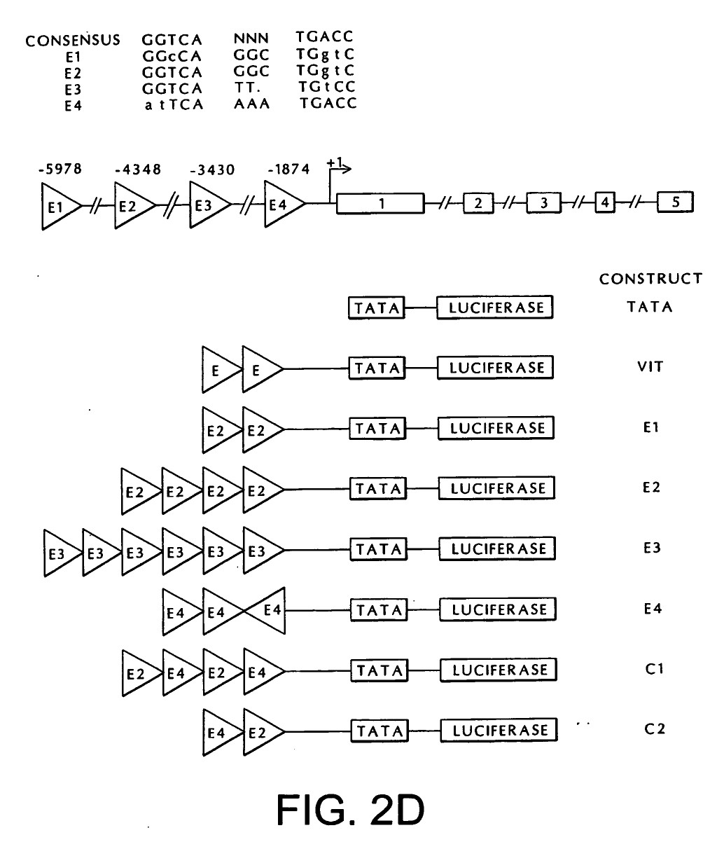

To determine if the induction of EIT-6 by estrogen is estrogen receptor-mediated, the effect of estrogen antagonists (4-hydroxy-tamoxifen and ICI 182,780) on EIT-6 mRNA levels was determined. BT-474 cells were cultured with ICI 128,780 (1 μM) or 4-hydroxy-tamoxifen (10 μM) for 6 hours prior to estrogen (10 nM) treatment for an additional 24 hours. Consistent with our SAGE data, EIT-6 was induced only by estrogen, and this induction was completely and partially abolished by the addition of ICI 182,780 and tamoxifen, respectively (FIG. 1C). Thus, the increase in EIT-6 mRNA levels following estrogen treatment is both estrogen and estrogen receptor dependent. There are multiple mechanisms by which estrogen can regulate the expression of EIT-6, e.g., (1) directly by transcriptional regulation, (2) directly by influencing mRNA stability, or (3) indirectly through other transcription factors/signaling pathways. Northern blot analysis of EIT-6 mRNA extracted from ZR75-1 cells after various times of estrogen treatment indicated that EIT-6 expression is induced by estrogen at about the same time as cathepsin D, a known direct target of estrogen receptors (FIG. 2A). This result suggests that EIT-6, similar to cathepsin D, may also be a direct transcriptional target of estrogen receptors. Addition of transcription inhibitors completely abolished EIT-6 induction by estrogen indicating that increased EIT-6 mRNA levels are likely due to increased transcription (data not shown). To further investigate if EIT-6 is a direct or indirect target of estrogen receptors, EIT-6 mRNA levels were measured following estrogen treatment BT-474 cells in the presence of a protein synthesis inhibitor (cycloheximide) (FIG. 2B). The cells were treated with cycloheximide (10 μg/ml) for 16 hours in the presence or absence of estrogen. Separate cycloheximide and estrogen treatment each modestly increased EIT-6 mRNA levels, but estrogen treatment in the presence of cycloheximide led to a much stronger induction. These findings indicated that induction of EIT-6 by estrogen does not require new protein synthesis. Therefore, EIT-6 is likely to be a direct transcriptional target of estrogen receptor. However, the possibility that other proteins are involved in the transcriptional activation of EIT-6 is not excluded.

To further characterize the mechanism by which the estrogen receptor induces EIT-6, a fragment of the proximal EIT-6 promoter was tested for its ability to confer estrogen responsiveness on a luciferase reporter gene. The luciferase construct was generated by subcloning a BAC (bacterial artificial chromosome)-derived fragment of EIT-6 promoter region (approximately 5.5 kb in length) into pBR-pl-luc [Polyak et al. (1997)]. The cells were transfected using FuGene 6 (Roche, Basel, Switzerland) and treated with estrogen the day after transfection. On the day following estrogen treatment luciferase activity was determined using a luciferase assay system (Promega, Madison, Wis.). Measurement of luciferase activity in the cells following transient co-transfection of this construct with an expression construct containing a cDNA sequence encoding the estrogen receptor a demonstrated a modest but reproducible induction following estrogen treatment (FIG. 2C). The sequence of the above-described EIT-6 promoter region was analyzed and several potential estrogen responsive elements (ERE) closely resembling the consensus ERE sequence were identified (FIG. 2D). To determine whether these putative EREs could confer estrogen responsiveness, various constructs containing concatemers of each of these elements (in a variety of combinations) up-stream of a luciferase gene were generated (FIG. 2D). These concatamer constructs were generated by subcloning concatamers of PCR-derived fragments (of the EIT-6 promoter region) containing the putative ERE's into pBR-pl-TATA-luc [Polyak et al. (1997) Nature 389:300-305]. Luciferase and β-galactosidase activity was measured following transient co-transfection of these constructs with the estrogen receptor α-encoding construct (referred to above) and a vector containing a β-galactosidase-encoding cDNA sequence under control of a constitutive (CMV) promoter into HepG2 cells as described above and the values of luciferase activity were normalized for transfection efficiency by calculating ratios of luciferase activity to β-galactosidase activity which was measured using the Aurora GAL-XE reporter gene assay (ICN, Costa Mesa, Calif.). “Fold induction” was calculated according to the following formula:

Fold induction=(luciferase activity in estrogen treated cells÷βgalactosidase activity in estrogen treated cells)÷(luciferase activity in control cells÷βgalactosidase activity in control cells).

The data revealed that most of the putative ERE combinations demonstrated some, although relatively weak, estrogen responsiveness (FIG. 2E). The most marked (4-5fold) induction was observed using the E4 construct containing 3 copies of the E4 ERE. The E4 ERE is the closest of the four EREs tested to the transcription start site in the wild-type gene and contains a nearly perfect ERE amino acid sequence with only two mismatches compared to the consensus sequence (FIG. 2D). Two copies of the consensus ERE derived from the vitillogenin promoter (VIT) [McMahon et al. (1999) Gene Expr. 8:59-66] in a construct analogous to those described above led to a 10-11 fold induction in luciferase activity following estrogen treatment (FIG. 2E). Therefore, the 4-5 fold induction observed with the E4 ERE was significant and indicates that the E4 fragment is a functional ERE. Although these experiments do not prove that any of these putative EREs are necessary for the induction of EIT-6 by estrogen, they do show that these elements can confer estrogen responsiveness. Therefore, direct binding of estrogen receptors to these putative EREs could be responsible for the transcriptional induction of EIT-6.

### EXAMPLE 3.

### EIT-6 is a Nuclear Protein that Enhances Cell Proliferation

EIT-6 is a novel human gene and, although it is homologous to the SM-20 rat immediate-early gene induced by growth agonists, it is not the human orthologue of this rat gene (FIG. 3A). EIT-6 appears to encode a protein with evolutionarily conserved function: there is a C. elegans EIT-6 homologue (FIG. 3A and B) that was identified as an egg laying defective mutant (egl-9) [Trent et al. (1983) Genetics 104:619-647], and several additional SM-20/EIT-6 homologues were identified from various other species including several types of bacteria (FIG. 3B). The egl-9 protein was recently shown to have to have prolyl hydroxylase activity [Epstein et al. (2001)].

Immunohistochemical analysis of the rat SM-20 gene demonstrated cytoplasmic staining, while the human SM-20 orthologue, another related human gene (SCAND2), and EIT-6 all contain potential nuclear localization signals [Dupuy et al. (2000) Genomics 69:348-354; and Wax et al. (1994) J. Biol. Chem. 269:13041-13047]. The nuclear localization signal in EIT-6 has the sequence PEAPKRKWAE (SEQ ID NO:30) Most of the homology between EIT-6 and SM-20 resides in the C-terminal region with the N-terminal domain (containing the nuclear localization sequence) showing little to no homology. To determine the sub-cellular localization of EIT-6, a construct expressing a fusion protein (EIT-6-GFP), in which EIT-6 is fused to green fluorescence protein (GFP) was generated. The EIT-6-GFP-encoding cDNA fragment was generated by PCR and subcloned into the pShuttle-CMV vector [Flatt et al. (2000) Cancer Lett. 156.63-72; He et al. (1998) Proc. Natl. Acad. Sci. USA 95:2509-2514] to create pShuttle-EIT-6-GFP. MCF1OA cells were transiently transfected with pEGFP-N (containing only the GFP-encoding sequence), pEGFP-mito (BD Biosciences Clontech, Palo Alto, Calif.; containing a sequence GFP fused to a mitochondria-targeting peptide), or pShuttle-EIT-6-GFP and analyzed by fluorescence microscopy. Cells transfected with pEGFP-N and pEGFP-mito revealed predominantly cytoplasmic and mitochondrial, respectively, localization of fluorescence (FIG. 3C). In contrast, in cells transfected with pShuttle-EIT-6-GFP the EIT-6-GFP protein was detected only in the nucleus. Similar results were obtained by western blot analysis of fractionated cell extracts prepared from cells expressing a hemaglutinin epitope tagged EIT-6 protein. These findings indicated that EIT-6 is likely to be located and function in the nucleus.

In order to test whether EIT-6 expresssion influences cell proliferation and/or survival, colony assays were performed. An expression construct was generated by subcloning into pCEP4 (Invitrogen Corporation, Carlsbad, Calif.) a PCR derived fragment encoding human EIT-6 with a double hemagglutin tag fused to its C-terminus to create pCEP4-EIT-6. T47D ER+breast cancer cells were transfected with a control construct (pCEP4) or the pCEP4-EIT6 construct. Stable transfectants were selected by culturing the cells in the presence of hygromycin for two weeks after which colonies were visualized by crystal violet staining. The expression of EIT-6 was confirmed by immunoblot analysis of cell extracts prepared from pools of stable clones using antibody specific for HA (data not shown). The number of colonies per cm² was determined by spot-densitometry-assisted counting using a MultiImage Lightbox (Alfa Innotech, San Leandro, Calif.). Two flasks per experimental group and two independent areas in each flask were analyzed. The cells were grown in standard fetal bovine serum (FBS) (10%) supplemented RPMI medium. The cells were cultured in the presence or absence of estradiol (10 μM). In three independent experiments expression of EIT-6 led to a significant (3-4-fold) increase in colony numbers (FIG. 3D, first two bars). Similar results were obtained in MDA-MB435S ER negative breast cancer cells. These data indicated that EIT-6 overexpression enhances colony growth in human breast cancer cells whether or not the cells express the ER.

To test if expression of EIT-6 can confer estrogen independent growth, ER+and estrogen dependent T47D breast cancer cells were transfected with control pCEP4 or pCEP4-EIT6 constructs. Stable transfectants were selected by culturing the cells in phenol red-free medium supplemented with charcoal/dextran treated (and thus steroid hormone depleted) FBS (5%) and hygromycin. Half the flasks contained estrogen (10 nM). After two weeks of dry selection, colonies were visualized by crystal violet staining and counted as described above. Very few colonies were observed with control pCEP4 transfected cells in the absence of estrogen confirming the requirement of estrogen for T47D cell growth (FIG. 3D). In contrast, a significant number of colonies was observed in EIT-6 transfected cells in the absence of estrogen indicating that EIT-6 expression relieves estrogen dependency. Addition of estrogen increased colony numbers in both control pCEP4 and EIT-6 transfected cells and thus EIT-6 expression may not be sufficient to completely alleviate estrogen dependence.

It should be understood that various modifications can be made to the above-described embodiments without departing from the spirit of the invention. Accordingly, the invention is limited only by the following claims.

What is claimed is:

1. A method of identifying a compound that inhibits an activity of EIT-6, the method comprising:
(a) identifying a cell as expressing a functional EIT-6 molecule; and
(b) testing in vitro for the ability of a test compound to inhibit proliferation or survival of the cell,
wherein a compound that inhibits the proliferation or survival of the cell is a compound that can potentially inhibit the activity of EIT-6.

2. The method of claim 1 further comprising;
(c) providing a second cell, wherein the second cell does not substantially express a functional EIT-6 molecule; and
(d) determining in vitro if the test compound inhibits proliferation or survival of the second cell.

3. The method of claim 1, wherein the cell is transfected with or transformed with a DNA encoding a functional EIT-6 molecule.

4. A method of identifying a compound that inhibits an activity of EIT-6, the method comprising contacting a test compound with a functional EIT-6 molecule and determining whether the test compound inhibits an activity of the functional EIT-6 molecule.

5. The method of claim 4, wherein the activity comprises hydroxylation of a proline residue in a polypeptide.

6. The method of claim 4, wherein the activity comprises conversion of 2-ketoglutarate to succinate.

7. A method of inhibiting the activity of EIT-6, the method comprising:
(a) identifying a cell as expressing a functional EIT-6 molecule; and
(b) contacting the cell with a compound that inhibits an activity of EIT-6.

8. A method of inhibiting the activity of EIT-6 in a subject, the method comprising:
(a) identifying a mammal as having a cancer that expresses EIT-6; and
(b) administering to the mammal a compound that inhibits an activity of EIT-6.

9. The method of claim 7, wherein the activity comprises hydroxylation of a proline residue in a polypeptide.

10. The method of claim 7, wherein the activity comprises conversion of 2-ketoglutarate to succinate.

11. The method of claim 4, wherein the compound is a 2-oxoglutarate analog.

12. The method of claim 11, wherein the 2-oxoglutarate analog is N-oxalylglycine or dimethyl-oxalylglycine.

13. The method of claim 11 wherein the 2-oxoglutarate analog is N-oxalyl-2S-alanine.

14. The method of claim 7, wherein the compound is pyridine-2,5-dicarboxylic acid or an analog of pyridine-2,5-dicarboxylic acid.

15. The method of claim 7, wherein the cell is in a mammalian subject.

16. The method of claim 7, wherein the cell is a cancer cell.

17. The method of claim 16, wherein the cancer cell is a breast cancer cell.

18. The method of claim 7, wherein the cell is an estrogen responsive cell.

19. An isolated DNA comprising:
(1) a nucleic acid that encodes a polypeptide consisting of (a) SEQ ID NO:1 or
(b) SEQ ID NO:1 but with one or more conservative substitutions;. or
(2) the complement of the nucleic acid,
wherein the polypeptide has the ability to enhance the proliferation of a cell.

20. The DNA of claim 19, wherein the nucleic acid consists of SEQ ID NO:2.

21. A vector comprising the DNA of claim 19.

22. The vector of claim 21, wherein the nucleic acid is operably linked to a transcriptional regulatory element (TRE).

23. A cell comprising the vector of claim 21.

24. A cell comprising the vector of claim 22.

25. A method of making a polypeptide, the method comprising culturing the cell of claim 24 and isolating the polypeptide from the culture.

26. A polypeptide encoded by the DNA of claim 19.

27. A polypeptide comprising (a) a fragment of SEQ ID NO:1 or (b) the fragment but with one or more conservative substitutions, wherein the fragment and the fragment with one or more conservative substitutions each have the ability to enhance proliferation of a cell.

28. An isolated DNA comprising a nucleic acid sequence that encodes (a) a fragment of a polypeptide consisting of SEQ ID NO:1 or (b) the fragment but with one or more conservative substitutions, wherein the fragment and the fragment with one or more conservative substitutions each are at least 85 amino acids long and each have the ability to enhance proliferation of a cell.

29. A method of inhibiting expression of EIT-6 in a cell, the method comprising delivery to the inside of a cell of an antisense oligonucleotide that hybridizes to an
EIT-6 transcript, wherein the antisense oligonucleotide inhibits expression of EIT-6 in the cell.

30. The method of claim 29, wherein the cell is in a mammal.

31. The method of claim 29, wherein the delivery comprises introduction into the cell of the antisense oligonucleotide.

32. The method of claim 29, wherein the delivery comprises introduction into the is cell of a nucleic acid comprising a TRE operably linked to a nucleic acid sequence, wherein the nucleic sequence is transcribed in the cell into the antisense oligonucleotide.

33. The method of claim 29, wherein the cell is a cancer cell.

34. The method of claim 33, wherein the cancer cell is a breast cancer cell.

35. An antibody that binds to EIT-6.

36. The antibody of claim 35, wherein the antibody is a monoclonal antibody.

37. The antibody of claim 35, wherein the antibody is a polyclonal antibody.

38. A method of identifying a compound that inhibits an activity of EIT-6, the method comprising:
(a) providing a cell that is transfected with or transformed with a DNA encoding a functional EIT-6 molecule; and
(b) testing for the ability of a test compound to inhibit proliferation or survival of the cell,
wherein a compound that inhibits the proliferation or survival of the cell is a compound
that can potentially inhibit the activity of EIT-6.
